(19) United States
(12) Patent Application Publication
(10) Pub. No.: US 2022/0322930 A1
(43) Pub. Date: Oct. 13, 2022

(54) METHOD AND APPARATUS FOR EVALUATION AND THERAPEUTIC RELAXATION OF EYES

(71) Applicant: Sina Fateh, Sunnyvale, CA (US)

(72) Inventor: Sina Fateh, Sunnyvale, CA (US)

(21) Appl. No.: 17/658,633

(22) Filed: Apr. 8, 2022

Related U.S. Application Data
(60) Provisional application No. 63/201,052, filed on Apr. 9, 2021.

(51) Int. Cl. A61B 3/032; H04N 13/271; H04N 13/344; H04N 13/383; A61B 3/08; A61B 3/028

(52) U.S. Cl. CPC A61B 3/032; H04N 13/271; H04N 13/344; H04N 13/383; A61B 3/08; A61B 3/0285

(57) ABSTRACT

A base acoustic emission is directed at an eye, and a return acoustic emission is detected from the eye. Base and return acoustic emissions are compared and differences evaluated to determine a descriptor of intraocular pressure. Also, stereo content is provided to a viewer with vergence depth and/or other features selected to bias the eyes towards therapeutically useful positions, movements, focuses, etc. and/or away from harmful positions, movements, focuses, etc. Therapy may facilitate improvements in eye health through reduction of intraocular pressure, reducing mechanical insult to the optic nerve and/or other structures, reducing muscle strain in eye orientation muscles, reducing forces applied to the eye lens and/or within the associated muscles and ligaments, etc., to benefit glaucoma, myopia, etc. Stereo targets may be presented to align the eyes for other testing. Eye alignment, testing, and/or motion treatment may be combined as an “end-to-end” process.

### CROSS-REFERENCE TO RELATED APPLICATIONS

This application claims the benefit of U.S. Provisional Application Ser. No. 63/201,052 filed on Apr. 9, 2021, the contents of which are incorporated by reference for all intents and purposes.

### FIELD OF THE INVENTION

Various embodiments concern acquisition of information indicating the condition of eyes, and/or the therapeutic relaxation of eyes. More particularly, various embodiments relate to determining pressures and/or stresses on the eyes, and/or opposing such pressures and/or stresses through adjusting the physical conditions of the eyes.

### BACKGROUND

Stresses of various sorts in or on the eyes may be associated with certain medical conditions, and/or resultant symptoms of those conditions. For example, high intraocular pressure (sometimes abbreviated TOP) may be associated with glaucoma. While the intraocular pressure itself may or may not be the root cause of certain problems associated with glaucoma (e.g., progressive loss of vision), tracking intraocular pressure over time and/or acting to reduce intraocular pressure may avoid or reduce the severity of such problems. Similarly, physical damage to the optic nerve due to particular eye movements/positions may be associated with glaucoma, progressive deformation of the lens of the eye may be associated with extensive close-up focusing that applies long-term compressive stress to the lens, etc.

Approaches for determining such factors, e.g., intraocular pressure, stress to the optic nerve, compressive tension of the lens, etc., may depend on clinical equipment. For example, applanation tonometry may be utilized to measure intraocular pressure. Typically this may involve anesthetizing the eyes, applying a dye, and applying contact pressure to the eye to determine the amount of force necessary to flatten a portion of the cornea. The intraocular pressure then may be calculated from from that force. However, while such approaches may be effective they may not lend themselves to testing on a routine basis. For example, applanation tonometery may not be suitable for use by an individual patient who wishes to collect data daily, or at other specific intervals or times, in a non-clinical setting. If intraocular pressure monitoring is limited to a clinical setting, the intervals between data points may tend to be relatively long, e.g., weeks to months. Such monitoring may not reveal changes on shorter timescales, such as rapid increase or decrease, cyclical variation throughout the day, fluctuations due to particular activities, etc.

In addition, treatments for such stress-associated conditions, where treatments may exist at all, may depend on pharmaceutical intervention, and/or other interventions as may be considered aggressive (e.g., surgeries). Such treatments, while potentially effective, may present risks of side effects and/or other concerns.

### BRIEF SUMMARY OF THE INVENTION

This disclosure contemplates a variety of systems, apparatus, methods, and paradigms for determining various eye stresses, and/or for treating such eye stresses.

In one embodiment a method is provided that includes generating a base acoustic emission, acoustically communicating the base acoustic emission to a subject's eye, and acoustically receiving a return acoustic emission from the eye. The base acoustic emission and return acoustic emission are communicated to a processor, and are compared to identify an intraocular pressure descriptor for the eye. The processor then registers the intraocular pressure descriptor. The base acoustic emission may be communicated to the eye with a speaker of a head mounted display, the return acoustic emission may be received from the eye with a microphone of a head mounted display, and the processor may be in the head mounted display.

In another embodiment a method is provided that includes determining an intent eye configuration for a viewer, including the orientations of the left and right eyes, with that intent eye configuration corresponding with a reduction of the intraocular pressure of one or both eyes. The method includes determining an intent vergence depth for a visual target that corresponds with the intent eye configuration. Left and right stereo image fractions for the visual target are generated, and are displayed so as to enable resolution of the left and right stereo image fractions into the visual target by the viewer. The vergence depth of the left and right stereo image fractions is adjusted over time towards the intent vergence depth, such that responsive to resolving the left and right stereo image fractions the viewer's eyes are biased towards the intent eye configuration, so as to facilitate the reduction of intraocular pressure.

The method may include changing the vergence depth of the left and right stereo image fractions over time dynamically, proximate the intent vergence depth, such that in maintaining resolution of the left and right stereo image fractions by said viewer, the viewer's eyes are biased towards a dynamic eye configuration that is dynamically proximate the intent eye configuration. The method may include displaying the left and right stereo image fractions with a head mounted display, and changing the vergence depth of the left and right stereo image fractions with a processor of the head mounted display.

In another embodiment a method is provided that includes determining an intent eye configuration for a viewer's eyes that includes orientations of the left and right eyes, with the intent eye configuration corresponding to a reduction of intraocular pressure of. The method includes determining an intent vergence depth of a visual target corresponding with the intent eye configuration, determining an intent eye path of the visual target corresponding with the intent eye configuration, or both. Left and right stereo image fractions are generated for the visual target, and are displayed so as to enable a resolution of the left and right stereo image fractions by the viewer. The vergence depth of the left and right stereo image fractions may be changed over time towards the intent vergence depth, such that responsive to maintaining resolution of left and right stereo image fractions by the viewer the viewer's eyes are biased towards the intent eye configuration so as to facilitate reduction of the intraocular pressure; or the left and right stereo image fractions may be translated over time towards the intent eye path, such that responsive to maintaining resolution of left and right stereo image fractions by the viewer the viewer's eyes are biased toward the intent eye configuration so as to facilitate reduction of the intraocular pressure; or both.

The method may include changing the vergence depth of the left and right stereo image fractions over time dynamically about the intent vergence depth, such that responsive to maintaining resolution of left and right stereo image fractions the viewer's eyes are biased towards a dynamic eye configuration, that is dynamically proximate the intent eye configuration, so as to facilitate reduction of intraocular pressure. The method may include changing the target fraction path of the left and right stereo image fractions over time dynamically about the intent eye path, such that responsive to maintaining resolution of left and right stereo image fractions the viewer's eyes are biased towards a dynamic eye configuration, dynamically proximate the intent eye path, so as to facilitate reduction of intraocular pressure. The method may include displaying the left and right stereo image fractions with a head mounted display, and changing the vergence depth of the left and right stereo image fractions with a processor of said the mounted display, changing the target fraction path of the left and right stereo image fractions with the processor of the head mounted display, or both.

In another embodiment a method is provided that includes determining an intent eye configuration a viewer's eyes that includes the orientations of the eyes, wherein that intent eye configuration corresponds to the reduction of intraocular pressure. The method includes determining an intent vergence depth of a visual target corresponding with the intent eye configuration of said eyes, generating the visual target, displaying the visual target to the viewer, and translating the visual target such that responsive thereto the viewer's eyes bias towards the intent eye configuration so as to facilitate reduction of intraocular pressure. The method may include displaying the left and right stereo image fractions with a head mounted display, and translating the visual target with a processor of the head mounted display.

In another embodiment a method is provided that includes determining an intent eye configuration a viewer that includes an intent focus depth of the lenses of the viewer's eyes, with the intent eye configuration corresponding to a decrease in myopia. The method includes determining an intent focus depth of a visual target corresponding with the intent eye configuration, and determining the intent vergence depth of the visual target that corresponds with the intent focus depth. Left and right stereo image fractions are generated for the visual target, and are displayed with a vergence depth thereto so as to enable resolution of left and right stereo image fractions by the viewer. The vergence depth of the left and right stereo image fractions is changed over time towards the intent vergence depth, such that responsive to maintaining resolution of left and right stereo image fractions the viewer's eyes are biased towards the intent eye configuration, so as to facilitate the decrease in myopia.

The method may include determining an intent eye path of the visual target corresponding with the intent eye configuration, and determining an intent target path of the visual target corresponding with said intent eye path. The method may include displaying the left and right stereo image fractions with a target fraction position to enable resolution of left and right stereo image fractions by the viewer. The method may include changing the target fraction position of the left and right stereo image fractions over time towards the intent target path, such that responsive to maintaining resolution of the left and right stereo image fractions the viewer's eyes are biased towards the intent eye configuration, so as to facilitate a decrease in myopia.

The method may include displaying the left and right stereo image fractions with a head mounted display, changing the vergence depth of the left and right stereo image fractions with a processor of the head mounted display.

In another embodiment a method is provided that includes determining an intent eye configuration that corresponds to an intent test path for said eyes, determining an intent target path of a visual target corresponding with the intent eye configuration, and determining left and right intent display positions for left and right stereo image fractions for the visual target that correspond with with the intent eye configuration. Left and right stereo image fractions are generated for the visual target, and are displayed at display positions to enable resolution of the left and right stereo image fractions by the viewer. The display positions of the left and right stereo image fractions are changed over time towards the intent display positions, such that responsive to maintaining resolution of the left and right stereo image fractions the viewer's eyes are biased towards the intent eye configuration, so as to facilitate executing a test of said eyes. The method includes executing the test, and registering the test.

In another embodiment a method is provided that includes determining a first intent eye configuration for a viewer that includes a first intent eye configuration, corresponding to an intent test path for the eyes. The method includes determining a first intent target path of a first visual target corresponding with the first intent eye configuration, and determining first left and right intent display positions for first left and right stereo image fractions for the first visual target corresponding with the first intent eye configuration. The first first left and right stereo image fractions are generated for the first visual target and are displayed at first display positions so as to enable resolution of the first left and right stereo image fractions by the viewer. The method includes changing the first display positions of the first left and right stereo image fractions over time towards the first intent display positions, such that responsive to maintaining resolution of first left and right stereo image fractions the viewer's eyes are biased towards the first intent eye configuration so as to facilitate executing an acoustic test of said eyes.

The method includes generating a base acoustic emission, acoustically communicating the base acoustic emission to one of the eyes of the viewer, and acoustically receiving a return acoustic emission from the eye. The method includes comparing the base acoustic emission and the return acoustic emission so as to identify an intraocular pressure descriptor for the eye. The intraocular pressure descriptor is registered.

The method includes determining a second intent eye configuration for that includes second orientations of the left and right eyes, wherein the second intent eye configuration corresponds to reduction of intraocular pressure. The method includes determining a second intent vergence depth of a second visual target corresponding with the second intent eye configuration, determining a second intent eye path of the second visual target corresponding with the second intent eye configuration, or both. Left and right stereo image fractions are generated for the second visual target, and are displayed so as to enable resolution of the second left and right stereo image fractions by the viewer. The method includes changing the second vergence depth of the second left and right stereo image fractions over time towards the second intent vergence depth, such that responsive to maintaining resolution of second left and right stereo image fractions the viewer's eyes bias towards the second intent eye configuration, so as to facilitate reduction of intraocular pressure; or translating the second left and right stereo image fractions over time towards the second intent eye path, such that responsive to maintaining resolution of second left and right stereo image fractions the viewer's eyes bias toward the second intent eye configuration, so as to facilitate reduction of intraocular pressure; or both.

### BRIEF DESCRIPTION OF THE SEVERAL VIEWS OF THE DRAWINGS

Various objects, features, and characteristics will become more apparent to those skilled in the art from a study of the following Detailed Description in conjunction with the appended claims and drawings, all of which form a part of this specification. While the accompanying drawings include illustrations of various embodiments, the drawings are not intended to limit the claimed subject matter.

FIG. 1A shows an example apparatus for evaluating intraocular pressure disposed relative to an eye, in top-down view.

FIG. 1B shows an example apparatus for evaluating intraocular pressure disposed relative to an eye with a base acoustic emission therein, in top-down view.

FIG. 1C shows an example apparatus for evaluating intraocular pressure disposed relative to an eye with a return acoustic emission therein, in top-down view.

FIG. 2 shows an example method for evaluating intraocular pressure in an eye, in flow chart form.

FIG. 3A, FIG. 3B, and FIG. 3C show arrangements for biasing the eyes of a viewer towards various configurations, in top-down view.

FIG. 4 shows an example apparatus as may be useful for providing therapeutic eye treatment, in perspective view.

FIG. 5A and FIG. 5B show example arrangements of left and right image fields for presenting stereo content as may relate to therapeutic eye treatment.

FIG. 6 shows an example method for therapeutic treatment of at least certain forms of glaucoma, in flow chart form.

FIG. 7A and FIG. 7B show an example method for therapeutic eye treatment, again in flow chart form.

FIG. 8 shows an example method for eye testing, in flow chart form.

FIG. 9 shows an example method for eye testing, utilizing mono content, in flow chart form.

FIG. 10 illustrates an example of a computer network system, in which various embodiments may be implemented, in schematic form.

The figures depict various embodiments described throughout the Detailed Description for the purposes of illustration only. While specific embodiments have been shown by way of example in the drawings and are described in detail below, the technology is amenable to various modifications and alternative forms. The intention is not to limit the technology to the particular embodiments described. Accordingly, the claimed subject matter is intended to cover all modifications, equivalents, and alternatives falling within the scope of the technology as defined by the appended claims.

### DETAILED DESCRIPTION OF THE INVENTION

The figures depict various embodiments described throughout the Detailed Description for the purposes of illustration only. While specific embodiments have been shown by way of example in the drawings and are described in detail below, the technology is amenable to various modifications and alternative forms. The intention is not to limit the technology to the particular embodiments described. Accordingly, the claimed subject matter is intended to cover all modifications, equivalents, and alternatives falling within the scope of the technology as defined by the appended claims.

As an initial and non-limiting summary, certain approaches are presented as examples herein.

With regard to evaluations, intraocular pressure may be determined acoustically, by sending an acoustic base signal towards an eye (without necessarily making physical contact between the emitter and the eye), and monitoring an acoustic return from the eye (again without necessarily making physical contact between the eye and the receiver). Comparison of the base and return signals may provide an indication of the intraocular pressure. For example, considering the eye as a fluid-filled sphere, the internal pressure, surface tension, etc. may affect the transformation (if any) of an acoustic emission that is sent towards the eye. In colloquial terms, the process may be considered as a form of sonar. Regardless of whether intraocular pressure is in itself a cause of damage associated with glaucoma or a symptom of glaucoma, variations in intraocular pressure may be revealing as to the current state and/or progress of glaucoma.

With regard to therapeutic intervention, disposing eyes in a “rest” or “neutral” configuration may address various stresses on the eyes. For example, certain eye orientations, eye motions, etc. may be associated with the application of physical stresses on the optic nerve and/or other eye structures; those stresses in turn may be associated with glaucoma and vision loss therefrom. In orienting the eyes in a neutral position, where such stresses may be to at least some extent avoided, damage to the eyes may be reduced or even counteracted. In colloquial terms, the eye may be allowed to rest for a time, and that rest may help to avoid or even reverse vision loss. For illustrative purposes, an analogy may be drawn to placing wrists in a neutral for some period to counteract carpal tunnel syndrome (though it is not suggested that the comparison is exact); providing the relevant physical structures with an opportunity to rest, even briefly, may be useful.

Somewhat similarly thereto, as another example tension in the muscles that orient the eyes may apply compression to the eyes, thus potentially increasing intraocular pressure which may in turn contribute to eye damage and loss of vision from glaucoma. In orienting the eyes to a neutral position, the muscles that orient the eyes may be encouraged to relax, again providing what might be colloquially referred to as a rest. Such relaxation may oppose elevated intraocular pressure, etc.

As another example, the muscles used to distort the lens of the eye so as to vary the focus thereof may cause permanent deformation of the lens, permanent shortening of the focusing muscles, and/or similar. Whether due to the lens becoming biased towards a particular focus (e.g., close up focusing), the muscles becoming incapable of fully relaxing, etc., the ability of the eye to focus throughout its full nominal range may become restricted. More concretely, if the eye is biased towards a focus on close objects, the eye's capability to focus on distant objects may be diminished (e.g., the eye may be said to be myopic). Again, by reconfiguring the eyes towards a different state—to continue the example above, encouraging the focusing muscles to act as though focusing on a distant target—myopia and/or other conditions may be opposed or even reversed. (It is noted that while a relatively straightforward presentation is made for a potential problem as may be addressed, in practice eye focus and/or other eye concerns may be more complex than is described herein for purposes of example. For instance, eye focus may involve not only the contraction of the ciliary muscle but also the suspensory ligaments, and/or other factors. A comprehensive structural and/or medical analysis of the eye is not presented herein.)

So as to achieve such eye relaxation, the eyes may be encouraged to act as though focusing on distant objects by presenting visual targets that exhibit a vergence associated with large distance, e.g., “at infinity” in a visual sense. Presenting suitable targets with suitable vergence may be accomplished conveniently through the use of a stereo display, such as a head mounted display (HMD). While real-world distance might at least in principle be utilized, presenting specific distances, specific variations in distance, specific positions within the visual field, etc. may not be practical when the real-world distance associated with a particular vergence is greater than may be achieved within a given room. Artificial visual targets with well-controlled vergence may exhibit advantages in therapeutic applications. In more colloquial terms, a head mounted display (such as a virtual reality headset, an augmented reality headset, etc.) may present a target that appears in terms of vergence to be at a distance of (for example) 6 meters even if the viewer is inside an office cube, room, or other enclosed space where no wall is more than 2 meters away, may present that target so as to appear to oscillate between (for example) 3.5 and 4.5 meters away, etc.

It is emphasized that the therapeutic techniques under consideration may be used relatively briefly; it is not required that relaxation therapy for eye motion, eye orienting muscle tension, eye focusing muscle tension, etc. must be, or even necessarily could be, carried out continuously. Rather, rest periods with reduced or at least differing stresses may be therapeutically useful.

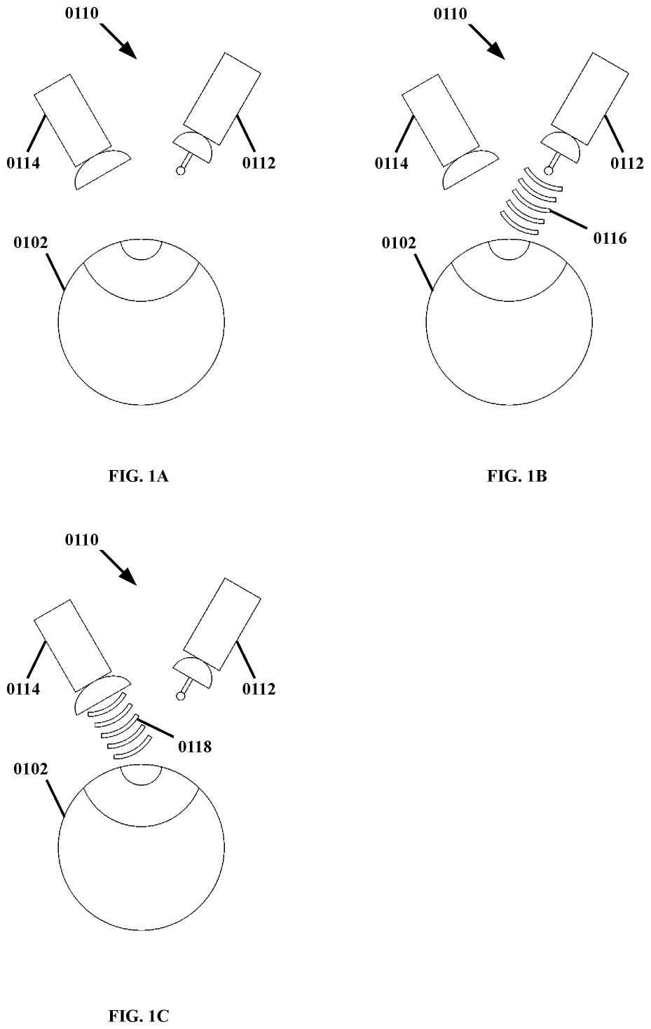

FIG. 1A depicts an example arrangement for evaluating intraocular pressure in an eye 0102. A test system 0110 includes an acoustic emitter 0112 and an acoustic receiver 0114. In the example shows, the acoustic emitter 0112 and the acoustic receiver 0114 are both oriented toward the eye 0102, each offset at an angle (as illustrated approximately 30 degrees). Such “corner to corner” approach may be useful in at least certain instances, but is presented as an example only. Variations thereon, e.g., different angles, different placements, etc. may be suitable. In addition, while the arrangement in FIG. 1A shows the acoustic emitter 0112 and acoustic receiver 0114 shown as aimed centrally to the front of the eye 0102 for illustrative purposes, other arrangements may aim elsewhere. For example, aiming acoustic emissions at the sclera may avoid various structures as may complicate acoustic returns, e.g., the cornea, lens, etc. Further, various arrangements may take advantage of certain acoustic “sweet spot” positions wherein acoustic returns are particularly strong and/or revealing. For example, the shape of the ocular cavity may focus acoustic returns at certain points, etc. The location and nature of such positions may vary depending on wavelength, amplitude, individual anatomy, etc., and are not limited. Additionally, while the arrangement in FIG. 1A presents an acoustic emitter 0112 and acoustic receiver 0114 that are not in contact with the eye 0102 or any other portion of the subject, contact sensing is not prohibited (e.g., an emitter and/or receiver in direct contact with the surrounding skin, the closed eyelid, or even at least potentially the eye itself). Similarly, semi-direct contact arrangements also may be suitable, for example wherein a cup, frame, or similar fixture may be disposed between the subject and the acoustic emitter 0112 and acoustic receiver 0114, such as for purposes of positioning and alignment. Mounting the acoustic emitter 0112 and acoustic receiver 0114 to such a structure, as may then fit to the subject, may for example facilitate repeatable positioning of the acoustic emitter 0112 and acoustic receiver 0114, accurate determinations of distance between the acoustic emitter 0112 and acoustic receiver 0114 and the eye 0102, etc. Other arrangements also may be suitable.

The orientation of the acoustic emitter 0112 and acoustic receiver 0114 are not limited. Moreover, while the acoustic emitter 0112 and acoustic receiver 0114 are shown for simplicity as being two distinct components, in other arrangements it may be suitable for a single element to function both to send and to receive acoustic emissions. Further, while only two elements of a test system 0110 are shown, the test system 0110 is not limited only thereto. For example, a test system 0110 may include a processor, other instruments, a display, etc. As a more concrete example, a test system 0110 may be or include a smart phone or similar device, e.g., with a speaker as may function as an acoustic emitter, a microphone as may function as an acoustic receiver, an on-board processor, etc. As another example, a smart phone or similar may function as part of a test system 0110, with other elements also being part of the test system 0110, e.g., a dedicated “plug in” acoustic emitter 0112 and/or a dedicated “plug in” acoustic receiver 0114. Other arrangements also may be suitable.

Turning to FIG. 1B, therein a portion of an operation of the test system 0110 therein is shown. As may be seen, an acoustic emitter 0112 and an acoustic receiver 0114 are disposed with respect to an eye 0102. The acoustic emitter 0112 may be seen to be producing a base acoustic emission 0116 directed towards the eye 0102. Though the base acoustic emission 0116 is shown in FIG. 1B as being directional and aimed, this is done for illustrative purposes. While a directional and/or aimed base acoustic emission 0116 is not prohibited, neither is such required. Thus the base acoustic emission 0116 may be omnidirectional, the acoustic emitter 0114 may not be required to be aimed at the eye 0102, etc.

Now with reference to FIG. 1C, another portion of an operation of a test system 0110 is shown therein. As may again be seen, an acoustic emitter 0112 and an acoustic receiver 0114 are disposed with respect to an eye 0102. A return acoustic emission 0118 may be seen directed towards the acoustic receiver 0114. Intermediate between FIG. 1B and FIG. 1C the base acoustic emission 0116 shown in FIG. 1B may be understood to have interacted with the eye 0102, producing the return acoustic emission 0118 shown in FIG. 1C. (As noted with regard to FIG. 1B, while a directional and/or aimed return acoustic emission 0118 as illustrated is not prohibited, neither is such required.)

Typically though not necessarily, the return acoustic emission 0118 as shown in FIG. 1C may differ from the base acoustic emission 0116 as shown in FIG. 1B in some fashion. In colloquial terms, the return acoustic emission 0118 may be considered as being a “reflection” of the base acoustic emission 0116. However in practice the return acoustic emission 0118 may not be a simple reflection as such. Rather, the base acoustic emission 0116 may be modified in some fashion through interacting with the eye 0102. For example, considering the eye 0102 as a fluid-filled sphere, it may be understood that acoustic emissions interacting with the eye 0102 may be reflected (in whole or in part) but also may be focused, may exhibit multiple returns (e.g., one off the front of the eye and a second off the back of the eye), may be to some degree absorbed and/or damped, may be diffracted, etc. Typically a return acoustic emission 0118 will bear at least some resemblance to an associated base acoustic emission 0116, but the degree of similarity is not limited, nor are the transformations limited, for purposes herein.

Notably however, certain transformations as may manifest between a given base acoustic emission 0116 and a resulting return acoustic emission 0118 may carry information regarding physical properties of the eye 0102. For example, given an eye of at least approximately known dimensions and configuration (e.g., a “typical” human eye), the intraocular pressure—the pressure of fluid therein—may affect the manner and degree of transformation between the base acoustic emission 0116 and the return acoustic emission 0118. For example, the physical tension of the surface of the eye 0102 may be at least in part a function of the internal fluid pressure within the eye 0102, e.g., higher internal fluid pressure may produce greater tension of the surface. As may be understood, the tension of a membrane such as a drum head may at least partially determine the pitch of sound produced when that membrane is struck; similarly the tension of the structure of the eye 0102 may impact the return acoustic emission 0118.

For example, if a return acoustic emission 0118 were to be shifted in pitch from a base acoustic emission 0116, that shift may be at least in part a function of the tension on the eye 0102, and thus may be a function of the intraocular pressure of that eye 0102. This is an example only; the potential variations in modification of a base acoustic emission 0116 in yielding a return acoustic emission 0118 may vary greatly, depending at least in part on the particulars of the base acoustic emission 0116 as well as on the particulars of the eye 0102 in question. As another example, some portion of the base acoustic emission 0116 may be conducted around the eye along the tensioned surface thereof, with the speed of such conduction being a function of the tension; in such case a lag time between emission of the base acoustic emission 0116 and the return of that portion of the return acoustic emission 0118 may be indicative of the tension of the eye surface, and/or thus indicative of the intraocular pressure of the eye 0102.

As yet another example, a resonant frequency of the eye may be at least in part a function of the intraocular pressure therein. Thus, varying the frequency of the base acoustic emission 0116 to find a point at which the return acoustic emission 0118 is consistent with the eye 0102 being addressed at a resonant frequency may be suitable. As a related note, while in FIG. 1B and FIG. 1C the base and return acoustic emissions 0116 and 0118 are illustrated as discrete events, e.g., one pulse, this is illustrative only and is not limiting. As described above, the frequency of the base acoustic emission 0116 may be varied; likewise the base acoustic emission 0116 may be an ongoing signal rather than a pulse, may include multiple components (e.g., a continuous signal with pulses overlaid, multiple frequencies, etc.), and/or otherwise may vary. The base acoustic emission 0116 is not limited, nor is the return acoustic emission 0118 deriving therefrom.

It is noted that a lack of change also may be considered. For example, if a base acoustic emission 0116 does not exhibit some particular change in a return acoustic emission 0118 for intraocular pressures above some threshold value, then observing that the return acoustic emission 0118 is not different in that respect from the base acoustic emission 0116 also may be relevant. Thus, a lack of change may be considered as well as a presence of change.

Other arrangements also may be considered, so long as in comparing the base acoustic emission 0116 and the return acoustic emission 0118 some descriptor of the intraocular pressure may be determined therefrom. It is noted that a “descriptor” of intraocular pressure as the term is used herein does not necessarily imply a measurement of intraocular pressure. For example, if a given comparison of a base acoustic emission 0116 and a return acoustic emission 0118 were to reveal that intraocular pressure were rising or falling, such an arrangement may be sufficient. Even an arrangement that reveals only that intraocular pressure is either remaining stable or not, without even indicating a direction of change, may be of use. While accurate and/or precise measurements of intraocular pressure are not excluded, so long as some therapeutically useful information may be discerned therefrom, the nature of the descriptor is not limited.

As a more concrete example, it may be suitable for a descriptor to be a simple numerical value, such as “6”. The value itself may not represent a measure of intraocular pressure; it may not be 6 “of anything”, just 6. However, if an increase of that value to 9 (for example) may indicate a change in intraocular pressure (whether an increase, a decrease, or even a change in unknown direction), then such a numerical value may be suitable as a descriptor for purposes herein.

In addition, with regard collectively to FIG. 1A through FIG. 1C, while therein is illustrated an example wherein a return signal is an acoustic return signal 0118, other arrangements wherein a base acoustic emission 0116 produces some non-acoustic emission also may be suitable. For example, a base acoustic signal may produce visible vibrations on the surface of an eye 0102. Though not necessarily acoustic in nature, such visible vibrations (and/or other indications) may be suitable for consideration as a return signal herein.

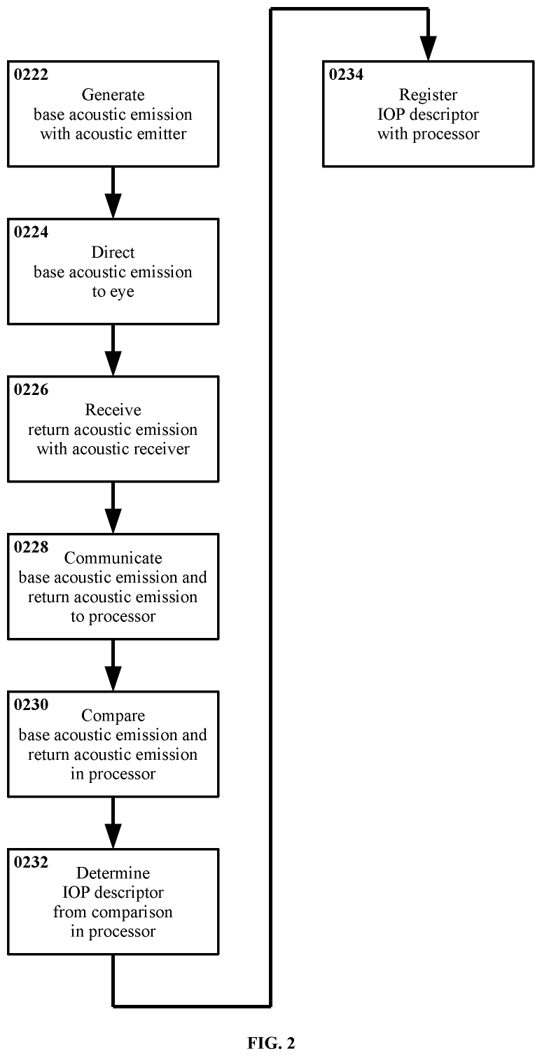

Moving on to FIG. 2, a method for evaluating intraocular pressure in an eye is shown therein, in flow chart form. In FIG. 2, a base acoustic emission is generated 0222 with an acoustic emitter. The precise nature, form, amplitude, etc. of the base acoustic emission is not limited; the precise nature, form, etc. of the acoustic emitter also is not limited. Typically though not necessarily, the base acoustic emission will be of a frequency, waveform, etc. as may be suitable for producing non-contact returns from a fluid-filled spheroid, at least analogous to sonar.

Continuing in FIG. 2, the base acoustic emission is directed 0224 towards an eye. In being so directed it is not required that the base acoustic emission be directed 0224 only towards the eye. For example, an omnidirectional base acoustic emission may be suitable. So long as the base acoustic emission may interact with the eye such that a return acoustic emission is available therefrom, arrangements are not limited. Also, while in practice the actions of generating 0222 an acoustic emission and directing 0224 that acoustic emission may be viewed as (and arguably may be) a single event, FIG. 2 presents generation and direction as two steps for purposes of clarity.

Though not shown in FIG. 2, the base acoustic emission typically may be considered to interact in some fashion with the eye. To use sonar as an analogy, an acoustic “ping” may “reflect” from the eye, with that reflection then being detected in some form as a return acoustic emission. As noted previously herein the return acoustic emission need not be a simple reflection, and the base acoustic emission need not be a simple ping (though such arrangements are not excluded).

Continuing in FIG. 2, the return acoustic emission is received 0226 with an acoustic receiver. The base acoustic emission and the return acoustic emission are communicated 0228 to a processor. The nature of the communication and the processor are not limited, though typically digital communication to a digital processor may be suitable. As noted previously herein, an acoustic emitter and an acoustic receiver may be in communication with and/or components of a larger device such as a smart phone or similar, in which case the smart phone or other device may communicate and/or process such information internally.

The base acoustic emission and return acoustic emission are compared 0230 in the processor. The precise nature of the comparison may depend at least in part on the particulars of the acoustic emissions, the anticipated difference(s) between the base and return acoustic emissions, etc. Typically though not necessarily some algorithm may be used to identify features of the return acoustic emission as differ from the base acoustic emission, and/or to correlate such differences, etc.

An intraocular pressure descriptor is determined 0232 from the comparison 0230 of the base and return acoustic emissions in the processor. As noted previously herein, the descriptor may be a measurement of intraocular pressure, and/or may be some non-measurement indication that intraocular pressure has or has not changed, has risen or fallen, etc. Thus the descriptor may be a pressure value, but alternately or in addition may include other information.

Still with reference to FIG. 2, the intraocular pressure descriptor is registered 0234 with the processor. Registration is not limited; registration may include a variety of actions and/or events, such as recording in a data store the intraocular pressure descriptor, the intraocular pressure itself (if known), the acoustic emissions, differences thereof, the time of the evaluation, the person whose eyes are being evaluated, the person carrying out the evaluation, etc. In addition or instead, registration may include but is not limited to displaying the descriptor and/or other information, communicating with an external system such as a medical database and/or a research facility, contacting a caregiver and/or medical personnel, activating an indicator (e.g., a caution light on the test system if the intraocular pressure evaluation may be of concern), etc.

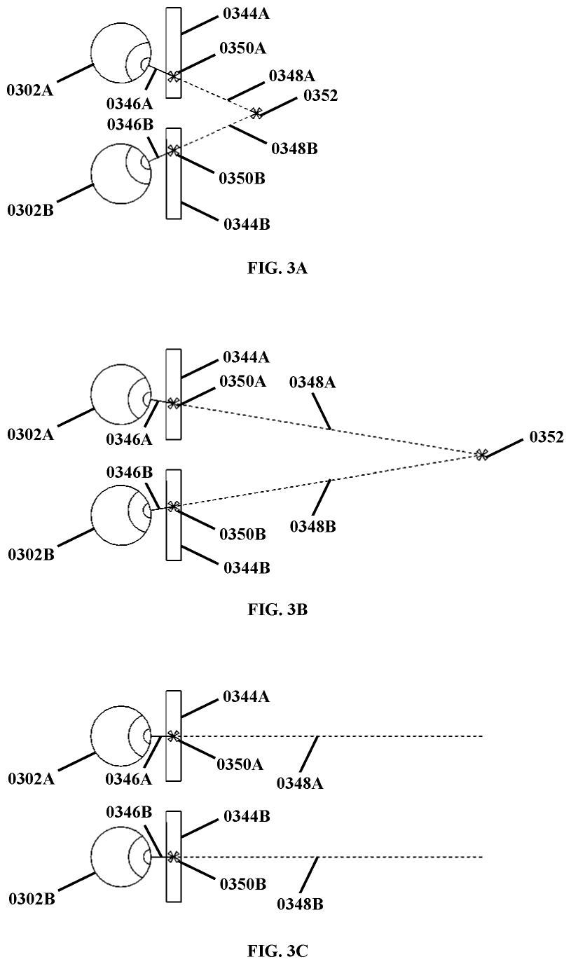

Now with reference to FIG. 3A through FIG. 3C, an arrangement showing the alignment of a subject's eyes with virtual objects at various virtual depths is illustrated. Referring specifically to FIG. 3A, a subject's left and right eyes 302A and 302B are shown. In front of the eyes 302A and 302B are left and right displays 0342A and 0342B in a stereo configuration. Such an arrangement may be brought about with a stereo head mounted display such as a virtual reality headset or an augmented reality headset (whether dedicated or modifying some other device such as a smart phone), etc. On the respective left and right displays 0342A and 0342B there are left and right stereo target fractions 0350A and 0350B. Left and right sight lines 0346A and 0346B may be seen extending from the eyes 302A and 302B to the target fractions 0350A and 0350B on the left and right displays 0342A and 0342B. In addition, left and right virtual sight lines 0348A and 0348B may be seen extending through the left and right displays 0342A and 0342B, converging on a virtual target 0352 some distance beyond the left and right displays 0342A and 0342B. Given the positions of the target fractions 0350A and 0350B on the displays 0342A and 0342B, when the user directs their attention to the target fractions 0350A and 0350B the visual appearance may be produced of the virtual target 0352.

As may be seen, the eyes 302A and 302B are angled inward towards one another; this angling may be referred to as vergence. The vergence that a viewer must exhibit in order to resolve the visual inputs from their left and right eyes 0302A and 0303B is a function of the distance to whatever target the user is attempting to resolve. Thus, the amount of vergence may used by the brain as an indication of the distance to the target in question, and may be useful in depth perception. Typically such distance may be carried out at an unconscious level. In presenting target fractions 0350A and 0350B in appropriate positions on displays 0344A and 0344B in a stereo configuration, an appearance of depth may be produced for a virtual target 0352 that is much greater than the physical distance to the displays 0344A and 0344B.

Now with reference to FIG. 3B, another arrangement at least somewhat similar to that in FIG. 3A is shown. However, as may be seen in FIG. 3B the depth to the virtual target 0352 is significantly greater than in FIG. 3A. As also may be seen, the angling of the left and right eyes 0302A and 0302B (e.g., the vergence) less in FIG. 3B than in FIG. 3A, and the positions of the left and right target fractions 0350A and 0350B are closer to the centers of their respective displays 0344A and 0344B.

Turning to FIG. 3C, yet another arrangement at least somewhat similar to FIG. 3A is shown. Again however, the vergence of the left and right eyes 0302A and 0302B is even less in FIG. 3C than in either FIG. 3A or FIG. 3B, and the positions of the left and right target fractions 0350A and 0350B approximately centered in their respective displays 0344A and 0344B. Indeed, the vergence shown in FIG. 3C is at least approximately zero; the sight lines 0346A and 0346B and the virtual sight lines 0348A and 0348B do not converge, instead being at least approximately parallel. The distance to a virtual target for such an arrangement would be effectively infinite (thus no such virtual target may be seen in FIG. 3C).

As may be understood through a comparison of FIG. 3A, FIG. 3B, and FIG. 3C, the apparent depth to the virtual target 0352 may be adjusted by moving the target fractions 0350A and 0350B. While the user visually tracks that virtual target 0352, the vergence of their eyes 0302A and 0302B will vary accordingly with the apparent depth. Thus, the angular orientation of the user's eyes 0302A and 0302B may be biased towards selected configurations. Engaging a user with a virtual target at infinite or near-infinite distance as in FIG. 3C may encourage a user to dispose their eyes 0302A and 0302B in the configuration shown in FIG. 3C, e.g., directly straight ahead.

In practice, human eyes do not function in a geometrically perfect or absolute manner, and thus the arrangements in FIG. 3A, FIG. 3B, and FIG. 3C should be understood as illustrative and not as necessarily precise models of real world eye configurations. Nevertheless, at least to a degree a user's eyes 0302A and 0302B may be biased towards selected configurations, through approaches as shown in FIG. 3A, FIG. 3B, and FIG. 3C.

In addition, it is noted that presenting target fractions 0350A and 0350B as shown does not necessarily control eye position, per se. The user may consciously choose to track such target fractions 0350A and 0350B or not. Thus, no absolute link is proposed; presenting and/or moving target fractions 0350A and 0350B to a user does not compel that user to track those target fractions 0350A and 0350B or to adjust the orientation of the user's eyes 0302A and 0302B. However, the human visual system is such that targets may be made naturally eye-catching, e.g., by moving or animating target fractions 0350A and 0350B, by displaying target fractions 0350A and 0350B prominently against a blank, uniform, or otherwise visually uninteresting background, by changing the apparent depth of the combined virtual target 0352, etc. Thus a useful degree of bias may be provided to encourage a user to track the content of interest, so as to reorient the user's eyes 0302A and 0302B in a therapeutically positive manner. Such encouragement may not even be apparent to the user on a conscious level, e.g., humans tend to track moving objects reflexively. Given suitable content suitably presented, the reorientation of the user's eyes 0302A and 0302B may require little or no effort or positive action on the part of a user, aside from the choice to make use of the approach in the first place. In colloquial terms, the eyes may be said to behave automatically when presented with appropriate content.

It is noted that for explanatory purposes, FIG. 3A, FIG. 3B, and FIG. 3C show simple arrangements with a static geometrical virtual target 0352 on-center with the viewer's eyes 0302A and 0302B, moving linearly outward. While such simple configurations are not prohibited, the range of possible motions and/or other changes is not limited. For example, a virtual target 0352 may be displayed off-center, may move vertically and/or horizontally, may vary in apparent depth, may be animated to change shape, size, etc., may be colored, may display changes in shade and/or may be fogged at different apparent depths, etc.

Related to such variations, particularly to vertical and/or horizontal motion within the user's field of view, it will be understood that the orientation of each eye 0302A and 0302B may be biased independently of one another to at least some degree. That is, with a virtual target 0352 centered in the field of view the inward vergence of each eye may be anticipated to be at least approximately equal in magnitude (though opposite in direction). However, for a virtual target to the left of center the right eye 0302B typically would be oriented leftward to a greater degree than would the left eye 0302A, and vice versa. Since human eyes typically tend to work together in a coordinated manner (e.g., to align both eyes with a target of interest) it may not be possible (or even necessarily desirable) to orient a user's eyes in a completely independent fashion. However, some degree of difference in orientation between left and right eyes 0302A and 0302B may be encouraged through suitable placement of target fractions 0350A and 0350B. In addition, to at least some degree such independence of orientation may be facilitated by blanking one or the other of the displays 0344A and 0344B so that the corresponding eye receives no input and thus has nothing on which to focus.

Thus with respect to FIG. 3A, FIG. 3B, and FIG. 3C, it may be understood that at least to a degree the orientations of the eyes 0302A and 0302B may be, if not necessarily controlled per se, at least biased towards desired configurations. Similarly, by biasing towards varying orientations over time, the eyes 0302A and 0302B in effect may be biased towards desired motions. Likewise, the eyes may be biased away from undesired orientations and/or motions.

What precisely may constitute desirable and/or undesirable orientations and/or motions of human eyes in a therapeutic sense may vary. For example, certain orientations and/or motions of the eyes may cause, aggravate, or at least contribute to certain eye health concerns. As a more concrete example, the optic nerve and associated structures are disposed generally at the back of the eye. Certain positions, motions, and/or speeds of motions may apply static and/or dynamic physical stresses to the optic nerve. While individually such stresses may appear insignificant, with repetition such stresses may prove to be problematic. Damage to the optic nerve may occur in a process at least somewhat analogous with repetitive stress injuries such as carpal tunnel syndrome, e.g., any single motion or position may be innocuous but sufficient repetition over time without rest or therapy may result in significant, potentially lasting harm. There is at least some reason to suspect that at least some cases of glaucoma may be driven not by excessive intraocular pressure, or at least not by intraocular pressure alone, but by ongoing mechanical stress to the optic nerve. For example, such a cause may explain cases wherein the visual degradation typical of glaucoma may be present but intraocular pressure appears normal. Thus, therapy to avoid harmful orientations and/or motions, and/or to encourage beneficial orientations and/or motions, may be of use in addressing glaucoma in at least certain patients.

As another example, eye orientation is controlled by a series of muscles attached to the eye and the surrounding tissue. Contraction of such muscles causes the eyes to shift their orientation up, down, left, right, etc. However, as in other muscles in the human body, the contraction of one muscle does not inherently cause an opposing muscle to fully relax. Various body muscles may retain some degree of tension, even when not actively reorienting the structures to which those muscles are attached. This may be true of muscles that control eye orientation as well, in at least some cases. Thus, one, several, or all muscles responsible for eye orientation may be at least somewhat in tension even when the eye is nominally stationary. Such tension in the eye muscles may increase or at least contribute to the increase of the intraocular pressure, e.g., tension in muscles connected to the eye applies mechanical compression to the eye, increasing the pressure therein. (The effect may be at least somewhat analogous to squeezing a water filled balloon, though the structures and forces are not necessarily identical.) Given that increased intraocular pressure may be associated with glaucoma, the potential may exist for tension in the muscles controlling eye orientation to aggravate, contribute to, and/or cause glaucoma in at least some patients. Conversely, reducing tension in the muscles controlling eye orientation may be therapeutically useful in addressing glaucoma, in at least such patients. It is noted that in at least certain eye surgeries, temporarily severing certain muscles controlling the eye may reduce the intraocular pressure; in effect, removing (or at least reducing) the applied tension may reduce the internal pressure.

As another example regarding potential desirable and/or undesirable eye orientations and motions, the focal depth of the eye is adjusted by muscles surrounding the lens that, when contracted, change the shape of the lens. The orientation of the eye does not absolutely control the contraction of the lens shaping muscles. However, given a target at a certain apparent depth, there is a natural tendency to focus the eye to that depth. Thus, given stereo targets displayed at a suitable depth, a bias may be applied to the lens shaping muscles. For example, a virtual target is presented at a given apparent depth, the eye orienting muscles orient the eyes orient to a vergence suitable for that depth, and the lens shaping muscles act in coordination with the eye orienting muscles to shape the lens to a suitable form for focusing on an object at that depth. However, certain behaviors may cause potentially lasting changes to the eye, that may reduce the eye's ability to focus across a full range of depths. When a person looks at a close target, the eye shaping muscles contract strongly to deform the lens to exhibit a short focal length. If a person looks at close targets for long periods, the eye shaping muscles may develop a bias towards compression, potentially becoming less capable of fully relaxing (and thus of enabling focus on distant objects). Likewise, the lens itself may undergo a degree of lasting deformation, potentially becoming less capable of returning to a shape suited for focus on distant objects. In either case, distance vision may suffer.

It is noted that certain practices that are currently common may contribute towards issues referenced in the preceding examples. For example, use of a smart phone or similar device, wherein the screen is held close to the eyes, may require a high degree of vergence of the eyes and/or a high degree of compression of the eye lens by the lens shaping muscles. Similarly, the use of computer monitors also may require a high degree of vergence and/or lens compression (though perhaps not such a high degree as with a smart phone). In both cases, it may be common for large numbers of persons to use such devices for long periods of time, e.g., tying on a laptop computer for hours at a time, texting on a smart phone for hours, etc. While a certain and absolute causative link between such practices and various eye health conditions is not proposed herein, and is not required, the potential risks may be non-trivial in scope and/or severity.

As noted previously, typically muscle actions associated with vision, and coordination therebetween, may be unconscious, e.g., positive action may not be required on the part of a viewer to make the orienting muscles in their left and right eyes to coordinate so that both eyes exhibit suitable vergence for a target at a given depth, or to make lens shaping muscles contract, relax, etc. However, that very unconscious nature of such actions may in at least certain cases present difficulties. A muscle that does not require conscious control may in at least certain circumstances be resistant to conscious control. Deliberately selecting a specific configuration and/or degree of tension in the eye orienting muscles, lens shaping muscles, etc. may prove difficult for at least many patients. As a more concrete example, it may be challenging to align and/or focus the eyes to “infinity” when in a confined space such as an office cubicle or a small apartment room, wherein every object within the visual field is much closer than infinity, e.g., a few meters away or less.

Thus, while in the abstract it may be a simple matter of relaxing the appropriate muscles to avoid potential eye health issues, in practice “just relax” may be of limited usefulness without some supporting therapy. Likewise, in principle pharmaceutical agents exist that may assist in relaxing certain eye related muscles and/or other tissues, e.g., belladonna and/or extracts thereof may be used for such applications. However, the long term, regular use of such pharmaceutical agents may not be well understood, and even when well understood may present risks of significant side effects. (For example, belladonna may be toxic in certain doses, and the effects of routine use over long periods may not be well documented medically.)

However, to return to the arrangements in FIG. 3A, FIG. 3B, and FIG. 3C, with a stereo display, a bias may be applied to a user's eyes 0302A and 0302B through presentation of suitable stereo content, e.g., target fractions 0350A and 0350B that correspond with a virtual target 352 at a given apparent depth. By selecting, varying, and/or controlling that apparent depth so as to bias the eyes towards beneficial eye orientations, movements, etc. and/or away from undesirable orientations, movements, etc., it may be possible to provide therapeutic benefit to at least some users for at least certain medical conditions, including but not limited to glaucoma and myopia.

Typically though not necessarily, therapy as considered herein may be at least broadly understood as opposing the factors as may be causing the difficulties. For mechanical-interference issues with the optic nerve bundle, therapy may include but is not limited to providing periods of reduced mechanical stress by orienting and/or moving the eyes in a manner that does not contribute to such mechanical interference and/or associated damage. For eye orienting muscle tension as may contribute to increased intraocular pressure, therapy may include but is not limited to relaxing tension in the relevant muscles by orienting the eyes in a “neutral” position as may facilitate low stress in and/or relaxation of those muscles. For lens compression as may contribute to myopia, therapy may include but is not limited to relaxing contraction of the lens shaping muscles so as to relieve tension therein and/or reduce applied forces to the lens, e.g., through presenting visual content at apparent (vergence based) distances that would correspond with a long focus of the eye lens. In certain cases therapy may include or be a form of training, e.g., leading a user in practicing non-damaging eye motions with regard to mechanical stress on the optic nerve, so as to encourage the user in making such motions habitual. However, these are examples only, and other arrangements also may be suitable.

In addition, it is noted that the precise conditions, orientations, motions, etc. may vary due to a number of factors, including the specific user, their particular medical concerns, etc., and are not limited herein. Likewise, the degree of benefit (if any) from a given therapeutic visual program may vary as well, and also are not limited herein. Similarly, the degree of efficacy, the duration of effect, the frequency and duration of therapy sessions, etc. may depend on a wide range of variables, and are not limited herein.

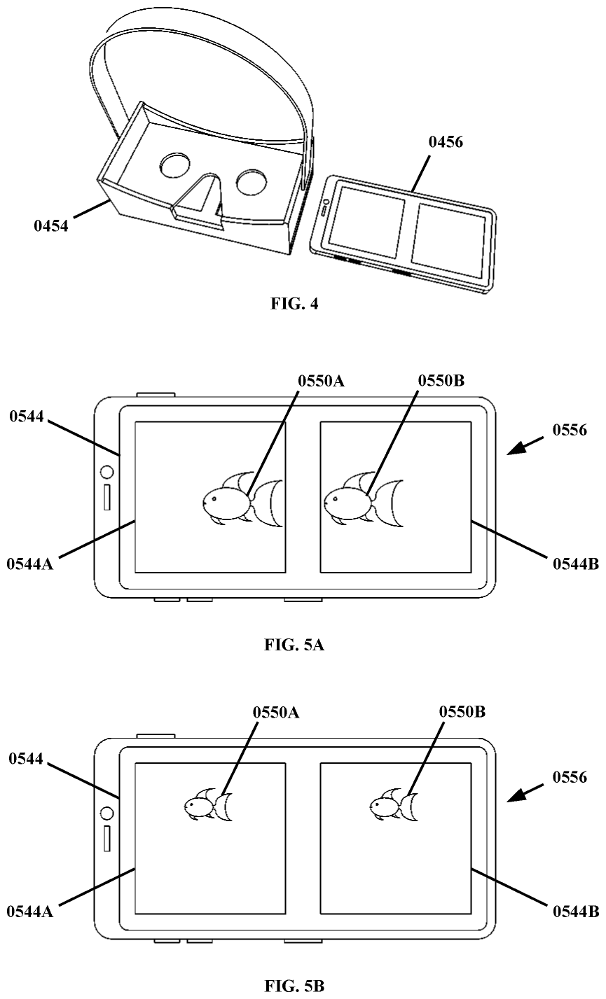

Turning now to FIG. 4, therein is shown an example apparatus as may be useful for providing therapeutic eye treatment. As illustrated in FIG. 4 the apparatus includes a head mounted viewer 0454 and a smart phone 0456. As may be understood the smart phone 0456 may be inserted into the head mounted viewer 0454 (e.g., sliding into a slot shaped to accommodate the smart phone 0456) such that the display of the smart phone 0456 may present content to a viewer in a stereo configuration. The smart phone 0456 is shown with two blank approximate squares on the display thereof, so as to approximate for illustrative purposes the portions of the display as may be utilized for left and right stereo fields.

An arrangement as shown in FIG. 4 may be useful, in that smart phones 0456 may be widely available in at least certain potential groups of patients, and a suitable head mounted viewer 0454 may be constructed as simple devices. For example, a head mounted viewer 0454 may be injection molded plastic or even cardboard, with no power source, processor, display, or other active components. So long as elements for generating and controlling content may be provided from some other source, such as a smart phone 0456, a head mounted viewer 0454 may be largely or entirely inert, relatively inexpensive, etc. However it is emphasized that FIG. 4 is an example only; mechanisms for providing stereo visual content may vary greatly, and are not limited herein. Alternatives may include, but are not limited to, dedicated head mounted display devices such as dedicated/integrated virtual reality headsets and/or augmented reality headsets. In addition, other active or passive systems may be suitable, such as so-called 3D glasses utilizing two optical filters of different colors (e.g., red/blue or red/green), two polarized filters with polarization directions perpendicular to one another, active LCD screens that alternate between being opaque and transparent, etc. Certain such approaches may enable the presentation of suitable visual content using displays at some distance from the viewer's eyes, such as televisions, computer monitors, etc. In addition, certain such approaches may facilitate the use of a single display system to enable multiple individuals to follow a given therapeutic regimen. (If it were anticipated that work conditions in a particular place may tend to aggravate myopia, for example, group sessions for myopia therapy may be useful. For instance, such group sessions may assist patients in maintaining a regular regimen, and/or may have other benefits.) Other arrangements also may be suitable.

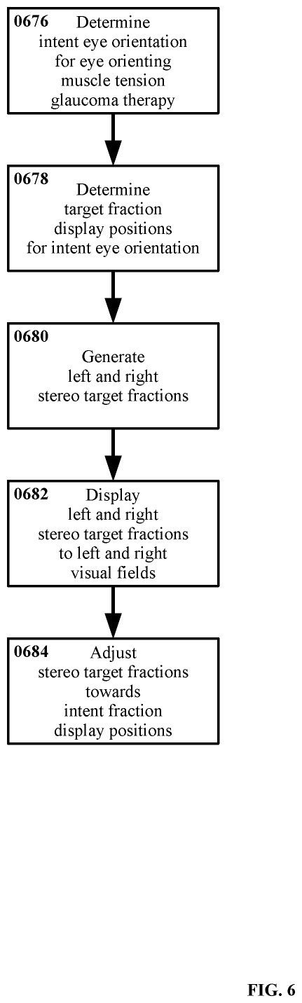

Moving on to FIG. 5A, therein is shown a smart phone 0556 with a display 0554. The display presents two approximate squares indicating left and right fields 0554A and 0554B for presenting stereo visual content. Such an arrangement may be suitable in certain instances, for example when utilizing a smart phone as shown previously in FIG. 4. However, as also noted previously with regard to FIG. 4, such an arrangement for presenting stereo content is an example only, and is not limiting.

As may be seen, in FIG. 5A the left and right fields 0544A and 0554B show left and right stereo target fractions 0550A and 0550B respectively. In the particular example of FIG. 5A, those stereo target fractions 0550A and 0550B take the form of views of a fish. As may be observed, in FIG. 5A the stereo target fractions 0550A and 0550B have a relatively small separation (the fish are close to the center of the screen 0554), and thus a target therefrom may be inferred to resolve at a relatively short apparent distance from the viewer (with reference made to FIG. 3A, FIG. 3B, and FIG. 3C).

Now with reference to FIG. 5B, an arrangement at least somewhat similar to that in FIG. 5A is shown, with a smart phone 0556 having a display 0554 and presenting left and right fields 0554A and 0554B thereon. Again, in FIG. 5B the left and right fields 0554A and 0554B show respective stereo target fractions 0550A and 0550B, again views of a fish. However as may be observed by a comparison of FIG. 5B with FIG. 5A, in FIG. 5B the stereo target fractions 0550A and 0550B are more widely separated and are closer to the top edges of the left and right fields 0554A and 0554B. It may be inferred that a target from stereo target fractions 0550A and 0550B in FIG. 5B may resolve with an appearance of greater distance than in FIG. 5A, and at a greater height. In more colloquial terms, the fish in FIG. 5B may be understood as having receded from the viewer, and also as having risen vertically.

Thus together, FIG. 5A and FIG. 5B may provide an illustration of certain aspects of content behavior as may relate to therapeutic functions. If FIG. 5A and FIG. 5B are considered as representing sequential moments in an animation, a viewer may first observe the fish at a relatively close depth, and then see the fish move upwards and recede to a more distant depth. To refer back to FIG. 3A, FIG. 3B, and FIG. 3C the eyes of a viewer concentrating on the fish may be biased towards shallower vergence angles, may focus the eye lenses to a greater distance, etc., with effects as already described herein and potential therapeutic benefits at least potentially deriving therefrom. It is again noted that the viewer would not have to concentrate on, for example, relaxing their eye positioning muscles, shifting their eyes to a “neutral” or otherwise therapeutically useful position, relaxing their lens focus muscles, etc. Rather, the viewer simply watches the fish. As the fish (or other target) changes apparent depth, a bias may be applied towards physiological effects (examples thereof having been described herein) as may be therapeutically beneficial, but the user need not even be conscious of any such effects. Thus the therapeutic process, beyond (colloquially) just watching the fish, may require no deliberate effort by the user, and may be considered as being user transparent.

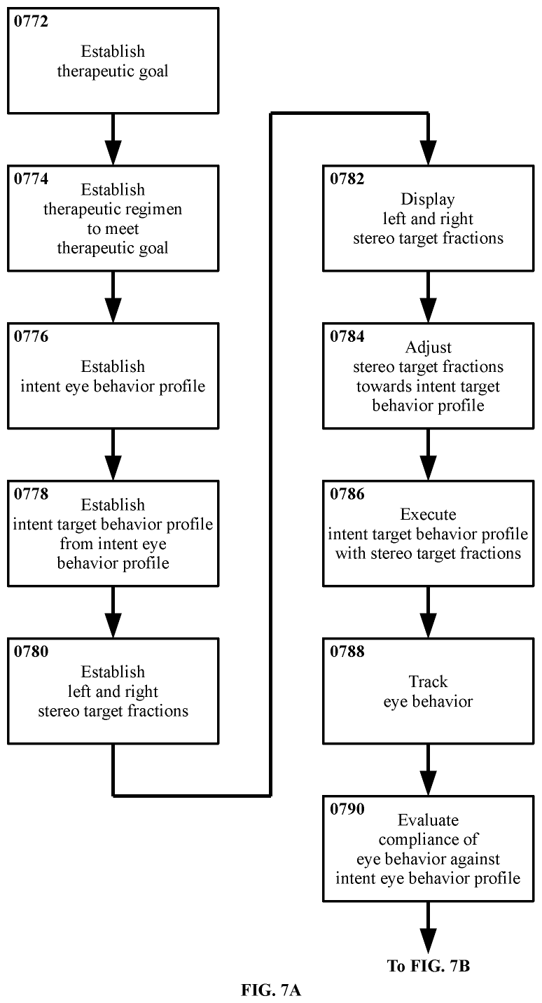

Turning to FIG. 6, therein is shown a method for therapeutic treatment of glaucoma, in flow chart form.

In FIG. 6, intent eye orientation is determined 0676 for therapeutically addressing eye orientation muscle tension as a potential factor in glaucoma. It is noted that step 0676 and indeed FIG. 6 as a whole is a specific example, and is not limiting; in practice the therapeutic arrangements presented in FIG. 6 may not apply to a patient with glaucoma unrelated to muscle tension in the eye orientation muscles (e.g., such method may be treating a condition that does not exist for that particular patient). However, for illustrative purposes FIG. 6 is directed to a relatively concrete example, wherein excess tension in the muscles that orient the patient's eyes is know or suspected to be causing, aggravating, etc. glaucoma in that patient. In such case, determining 0676 the intent eye orientation may be a matter of identifying a neutral configuration of the patient's eyes, e.g., an orientation thereof that exhibits a helpful level of muscle tension in the patient's eye orienting muscles (typically though not necessarily a reduced level of muscle tension). The particulars of the intent eye orientation may vary from one patient to another, based on the patient's individual anatomy, the physical condition of the eye orienting muscles, injuries, diseases, or other medical concerns, etc.

Display positions for left and right stereo target fractions are determined 0678 so as to be consistent with the intent eye orientation determination 0676 as described above. Given a particular configuration of the patient's eyes, where should target fractions be displayed to the patient viewing those target fractions in order to achieve or at least approach the intent eye orientation? Alternately, the matter might be considered as: what apparent depth should the target exhibit, and where in the viewer's visual fields should target fractions be presented to achieve that apparent depth? The target fraction display positions may vary from one patient to another just as may the intent eye orientation. In addition, other factors including but not limited to the particulars of the mechanism that will display the target fractions, e.g., different designs of head mounted displays may have screens of different sizes, at different distances, etc., and a system making use of a computer monitor may exhibit different optical geometries than a head mounted display, etc.

In colloquial and non-limiting terms, steps 0676 and 0678 may be understood as determining “what should the eyes do?” and “where should the content be placed to encourage the eyes do that?” Either or both determination may be applicable to multiple types of content, e.g., positioning a virtual fish or dolphin swimming in a virtual sea may be similar to positioning a virtual dragonfly flitting through a virtual forest, or a virtual aircraft in a virtual sky. The type, form, etc. of the target is not limited. Targets may be photorealistic, illustrative, or abstract, may be animated or static, color, grayscale, or black and white, may be of varying sizes and/or resolutions, etc. In addition, while certain examples herein may refer to targets presenting as real world elements, such as a fish, a dolphin, etc., other targets including but not limited to emojis, letters or numbers, etc. also may be suitable.

As a related matter, while only target content (what the viewer is supposed to look at to achieve therapeutic effects) is specifically addressed within FIG. 6, other content, whether stereo, flat, or otherwise, also may be considered, e.g., the target presented for therapeutic purposes may be a dolphin, but other creatures, objects, background features, etc. also may be made visible, and such additional elements are not limited.

Continuing in FIG. 6, left and right stereo target fractions are generated 0680. If the target is to be a fish, whatever graphical features are to present the appearance of that fish are generated 0680, typically though not necessarily by a processor (such as may be present in a smart phone, head mounted display, desktop computer, etc.). At least in principle target fractions may be pre-generated or otherwise provided, and the manner by which target fractions are made available is not limited.

The left and right stereo target fractions are displayed 0682 to the viewer's left and right visual fields, respectively. For example, considering an arrangement such as that shown previously in FIG. 5A and FIG. 5B, the fish therein may be displayed into the areas of the smart phone screen that are made visible to the left and right eyes respectively.

It is noted that, while target fraction display positions may have been determined 0678 already, it is not required that the target fractions be immediately displayed in the target fraction display positions (though this also is not prohibited). Rather, the target fractions may be adjusted 0684 towards the target fraction display positions, e.g., being first displayed 0682 in convenient and/or comfortable positions for the viewer and then being adjusted 0684 towards (and typically though not necessarily to) the desired target fraction display positions. In at least certain instances it may be uncomfortable or undesirable to immediately require the viewer to resolve a stereo target at an apparent depth, position, etc. as may be therapeutically useful; thus, the viewer may be “eased in” to, or at least towards, the desired eye configuration.

Similarly, while the target fractions may be maintained in therapeutically useful positions for some period of time (as may vary with the medical condition in question, the patient, etc.), it is not necessarily required that the target fractions be held precisely at the target fraction display positions at all times during therapy. Indeed, it may be useful in at least some cases to deliberately vary the apparent depth, position, etc. of the target being displayed. For example, with regard to the specific example of glaucoma related to eye orienting muscle tension in FIG. 6, it may be useful in relaxing the muscles in question to cycle the target through a range of depths, positions, etc., rather than following a more rigid a “snap to position and hold” approach. However, such particulars may vary greatly from patient to patient and embodiment to embodiment, and are not limiting herein.

While the arrangement in FIG. 6 is, as noted, presented as a concrete example for therapy for a specific eye condition with a specific approach, other approaches for the same condition and for other conditions also may be suitable. For example, in providing relief from ongoing insult to the optic nerve from certain positions or motions, a similar approach may be suitable. Likewise for training a user to avoid certain eye movements as may aggravate optic nerve damage, relieving stress in the eye focus muscles and/or the eye lens, etc.

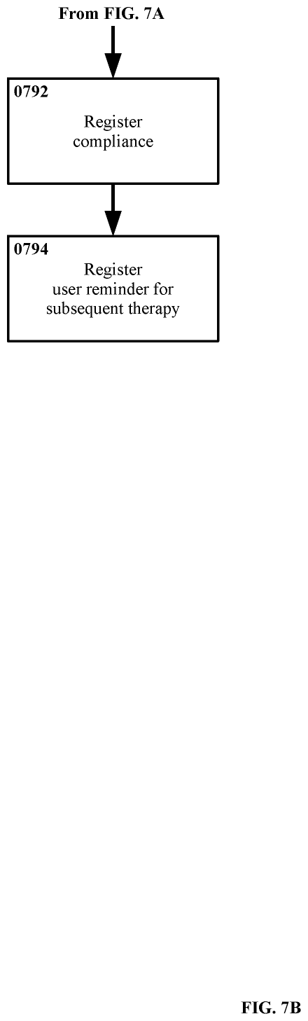

Now with reference to FIG. 7A and FIG. 7B, a somewhat more generalized (though still not necessarily exhaustive) example method is provided, again in flow chart form. Certain steps are presented in FIG. 7A and FIG. 7B as may have no one-to-one equivalent in FIG. 6. Such steps may be additions, may make explicit aspects as may be implicit in FIG. 6, etc. Not all methods necessarily will have all steps, nor necessarily in the precise order presented, nor are additional steps necessarily excluded.

Beginning in FIG. 7A, a therapeutic goal is established 0772. For example, a therapeutic goal may be to reduce muscle stress in the eye orientation muscles (similarly to what was presented with regard to FIG. 6), though other therapeutic goals also may be suitable. Also, therapeutic goals may be more detailed and/or specific, and/or may address other factors, e.g., reduce stress muscle stress in the eye orientation muscles by 25%, reduce intraocular pressure by 10%, etc., arrest progression of myopia such that an increase in correction is no more than 0.25 diopters five years, etc.

As noted not all methods necessarily will have all steps; for example depending on the particulars of execution, steps such as diagnosis may be considered, even though diagnosis is not explicitly shown in FIG. 7A. However, it is noted that given the data as may be obtained through approaches such as presented with regard to FIG. 2, such data may be applied to diagnosis, and/or to steps such as establishing 0772 the therapeutic goal. Additional or alternative approaches also may be suitable, however, including but not limited to diagnosis and/or setting of a therapeutic goal by an eye care professional or an automated diagnostic system, etc.

Continuing in FIG. 7A, a therapeutic regimen is established 0774 to meet the therapeutic goal. For example, an eye care professional may determine that sessions of 5 minutes duration focusing at a target nominally at infinity performed every 2 hours while using graphical displays may be suitable for a therapeutic goal of arresting progression of myopia. Other approaches may be suitable, such as standardized evaluation (e.g, following a medically validated table to determine the therapy, duration, frequency, etc.), use of algorithmic analysis based on patient data, or some other approach.

An intent eye behavior profile is established 0776. The eye behaviors as may be included in carrying out a given therapeutic regimen and/or to reach a given therapeutic goal are defined, determined algorithmically, or through some other approach. For example, the eyes must be brought to center, then gradually moved to neutral orientation, then cycled slowly into and out of that neutral orientation, then put through pan and tilt motions, etc. Step 0676 of FIG. 6 may be at least somewhat analogous; as previously noted with regard to FIG. 6 a colloquial description of step 0676 might be “what should the eyes do?” Where FIG. 6 was specific to reducing eye orienting muscle tension to address glaucoma, in step 0776 in FIG. 7A the question of “what should the eyes do?” may be considered quite broadly, for any of a variety of medical conditions and therapies. An intent eye behavior profile may be simple or extremely complex, but typically (though not necessarily) relates to eye orientation and/or motion, and/or the carrying out of other eye mechanical functions such as focusing, etc.

Still in FIG. 7A, an intent target behavior profile is established 0778, based at least in part upon the intent eye behavior profile. Where the intent eye behavior profile may address what the eyes do (e.g., orientation, motion, degree of focus, change in focus, etc.), the intent target behavior profile may address what a visual target to be presented to the viewer may do so as to encourage the viewer's eyes to move in such fashion. The intended target motion, etc., and thus the intent target behavior profile, may vary greatly, and may be simple or extremely complex. While motion, apparent depth, and size have already been referred to, it is emphasized that the factors as may be defined, controlled, and/or varied with respect to a visual target are not limited. For example, it may be that for certain cases changing the target color, opacity, contrast, shape, etc. may prove useful. Such variations may be specific to certain patients, to certain conditions (e.g., color may be relevant in therapy for some forms of glaucoma but not others), etc., and/or may not be well understood. If it were discovered (or even hypothesized), for example, that changing the shape of a target in some manner made myopia therapy more effective, whether or not an explanation of why target shape may be relevant could be determined (or even a certainty that therapy would be more effective with such shape changes), changing the shape could still be part of an intent target behavior profile.

While intent target behavior profiles may be tailored, individually generated for a given patient/condition, produced algorithmically in real time, etc., it may also be suitable for a given intent target behavior profile to be predetermined. For example, it may be that one or several “general purpose” presentations of a visual target may be suited for many or even all patients. Such general purpose presentations may be prepared in advance, e.g., being prerecorded and stored in processor memory, etc. Similarly, certain aspects may be predetermined while others are created in real time, for example a set of target motions may be prerecorded, while the target itself is selected or customized by a user, randomly generated, etc. (or vice versa). (It is noted that if an intent target behavior profile were prerecorded and widely used, such prerecording may occur well in advance of certain other steps that are numerically presented ahead of step 0778 in FIG. 7A; such may serve as an example, though not the only possible example, of a manner in which the ordering of steps may vary herein.) Regardless, the manner through which an intent target behavior profile may be established is not limited.

Continuing in FIG. 7A, left and right stereo target fractions are established 0780. Stereo target fractions typically though not necessarily may be derived from the intended appearance of the target itself as resolved by a viewer, e.g., if the viewer is intended to resolve the target as a dragonfly, the stereo target fractions that the viewer will perceive together as a dragonfly may be derived from a dragonfly image or model. The targets and thus also the target fractions as may be suitable are not limited, nor are any other features to be presented, such as backgrounds, distance effects (haze, fog, rain, blur, etc.), other foreground or background objects, and so forth. As with the intent target behavior profile, the stereo target fractions may be established 0780 in a variety of manners, such as being retrieved from stored memory, generated procedurally, selected by a viewer, created randomly, etc.

The left and right stereo target fractions are displayed 0782, typically though not necessarily to left and right respective visual fields within a display (though presentation on two separate displays or other options also may be suitable), such that a viewer may view the left stereo target fraction with the left eye and the right stereo target fraction with the right eye, and so resolve the fractions as a combined target with an apparent depth. As noted with regard to FIG. 6, while the stereo image fractions may be displayed 0782 immediately executing the intent target behavior profile, it also may be suitable for the stereo image fractions to be displayed 0782 “off profile”, for example in a location that is convenient and/or comfortable for the viewer to begin.

Continuing in FIG. 7A, the stereo target fractions are adjusted 0784 towards the intent target behavior profile. As noted above the stereo target fractions need not be displayed 0782 immediately carrying out the intent target behavior profile, however for therapeutic purposes typically it may be useful to follow or at least approach the intent target behavior profile. Thus, while perfect correspondence with the intent target behavior profile is not required, typically the stereo target fractions may exhibit a close correspondence therewith, and/or may be adjusted 0784 towards the intent target behavior profile.

The intent target behavior profile is executed 0786 with the stereo target fractions. Thus, at least nominally, the stereo target fractions follow the “flight plan” of the intent target behavior profile. However, even once following the intent target behavior profile, deviation therefrom may be suitable; should it be found that the viewer is not visually tracking the target, the stereo target fractions may be adjusted off-profile should such action be useful in addressing the matter (e.g., making it easier for the viewer to track the target, attracting the viewer's attention, etc.). A detailed if-then loop for such adjustments is not explicitly presented herein.

Continuing in FIG. 7A, eye behavior may be tracked. Typically though not necessarily eye motions are of interest, thus eye orientation tracking may be considered. Though eye focus may not strictly speaking be considered “eye motion” for purposes of motion tracking, in at least certain instances eye focus may be biased towards a planned set of behaviors at least in part by biasing the eyes to orient and move in particular manners, e.g., so that the eyes naturally focus to an apparent vergence depth of a target. Thus, eye motion tracking may be useful in evaluating focus behavior even if focus behavior is not directly determined. Tracking other information, including but not limited to saccadic motion, direct observation of focus, etc., also may be suitable. With regard to obtaining such data, it is noted that at least certain dedicated head mounted displays give consideration to eye tracking (regardless of the present degree of accuracy), and at least certain smart phones may have cameras suitable for eye tracking (whether or not eye tracking is presently implemented thereon). However, the use of additional sensors also is not prohibited, nor is the manner of eye observation nor the features observed limited.

As an aside, it is noted that, should eye tracking be performed, tracking eye behavior 0788 may be an ongoing process. Tracking thus may take place in parallel with certain other steps, e.g., in coordination with executing 0786 the intent target behavior profile. As noted, ordering of methods is not necessarily fixed to the specific arrangement illustrated for FIG. 7A.

Should eye tracking or other compliance data be obtained as part of the method (or is otherwise available), the compliance of the viewer's eye behavior may be evaluated 0790 against the intent eye behavior profile. Such evaluation may address the matter of whether the viewer's eyes moved and/or otherwise behaved as intended for the therapeutic session. Evaluation may vary in form and/or complexity. For example, a simple “fair/poor/good” standard may be utilized, an accuracy percentage may be determined, detailed information about how, when, and/or where the viewer's eyes did not move as anticipated/desired, etc.

Moving now to FIG. 7B, compliance with the regimen may be registered 0792 in some fashion. Registration may take many forms. For example, if the user simply “ran the program” for a particular therapy, this may be interpreted as having followed the regimen for that therapy. However, compliance may consider other factors, such as whether the user carried out therapy on schedule (at proper times and/or intervals, etc.), for the specified amount of time (if any), in the proper place/under specified conditions, whether the user met some minimum standard such as by being evaluated 0790, etc. In addition, registration may include other information, such as the time, date, user ID, specifics regarding the session (what images were used, etc.), and so forth. The manner of registration also may vary, including but not limited to recording relevant data in a data store (such as a storage on a smart phone or head mounted display), communicating data externally to some other system, forwarding data to a health care professional, care giver, etc., illuminating a tell tale (e.g., an LED is green when the user is compliant with a planned therapy regimen, yellow when marginally compliant, red when out of compliance), etc. Other arrangements also may be suitable.

Finally in FIG. 7A, a user reminder for subsequent therapy may be registered 0794. For example, a time for the next scheduled therapy session may be displayed in some fashion, an audible alarm may be sounded when the next session should be carried out, etc. Strictly speaking registering 0794 a reminder might be considered to be a special case of registering 0792 compliance, and it would not necessarily be unsuitable to combine the steps. However, it is noted that typically (though not necessarily) eye therapy may be an ongoing process, or at least a long duration process. Progressive myopia, glaucoma, etc. typically may be longstanding conditions; therapy, while at least potentially useful, may not necessarily cure the relevant conditions. Rather, arresting or reversing myopia, treating glaucoma, etc., may involve regular sessions for long periods, perhaps indefinitely. The degree of effect, the duration of effect, and/or other factors regarding various therapies as may be considered herein may not be well documented (at least in part due to such therapies not being previously available). Thus, therapy is not necessarily proposed as a short-term, definitive solution to eye concerns (though therapy as may behave in such manner is not excluded), but rather as an ongoing process as may include many repetitions on a regular or at least semi-regular basis.

The term “establishing” various elements as used herein should be understood broadly. In establishing an image, standard, regimen, motion path, etc., such elements may be loaded into a processor from stored executable instructions, may be defined as rule sets, may be calculated based on data and/or instructions, may be the result of the professional judgment of a medical professional, may be selected by the user, etc. It is noted that such options are not necessarily exclusive, and a given embodiment may utilize more than one. The manner by which features referenced herein may be established is not limited.

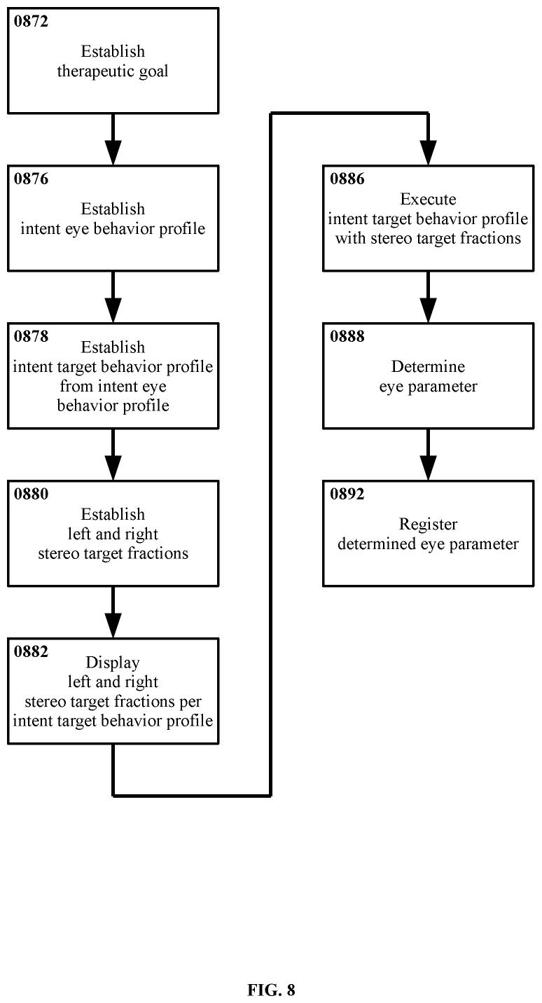

Now with reference to FIG. 8, an approach for testing one or more eye parameters is presented therein. Previously in FIG. 7A gathering data regarding the eyes was addressed in the form of tracking 0788 eye behavior while a user is conducting eye motion therapy. However, while information regarding how the eyes move may be useful for a variety of purposes (such as determining compliance with a treatment regimen, detecting potential eye problems based on eye movements, etc.), information as may be collected is not limited only to eye movement. Likewise, while information may be gathered during a course of treatment, as in FIG. 7A, data is not limited to being collected only during treatment. Indeed, embodiments may refer to testing, observing, etc. the eyes without treatment as such necessarily being performed at all (though combinations of treatment and/or testing are not excluded). FIG. 8 references an approach wherein visual targets may be presented so as to facilitate testing, e.g, aligning the eyes in a manner that is convenient for testing, particularly revealing, etc. Testing may include, but is not limited to, determinations of and/or regarding intraocular pressure. Other tests may include various forms of eye imaging, etc., and are not limited.

In FIG. 8, a therapeutic goal is established 0872. In the particular example of FIG. 8 the therapeutic goal may be to facilitate and/or carry out a determination regarding intraocular pressure, for example. In such case, establishing 0876 an intent eye behavior profile may include the subject's eyes being oriented in a particular manner, e.g., straight ahead, horizontally centered and inclined upward, vertically centered and panned left, etc. While such static eye orientations may be useful for certain tests, the behavior profile is not limited only to static configurations. For example, it may be desirable that an intraocular pressure measurement be conducted while the eye is panning left to right (e.g., related to effects of eye orientation muscle movements on intraocular pressure, the possibility of mechanical interference/insult regarding the optic nerve during certain eye motions, etc.). In such instance, an intent eye behavior profile may include panning left to right one or more times, so as to facilitate whatever testing may be of interest. Similarly, observations regarding focusing of the eye may be of interest in certain instances, in which case an intent eye behavior profile may include one or more changes in depth of focus.

Continuing in FIG. 8, an intent target behavior profile is established 0878 based on the intent eye behavior profile. The particulars of the intent target behavior profile will depend at least in part on the intent eye behavior profile. For example, as noted previously for certain tests the intent target behavior profile may include a static eye orientation. For an arrangement wherein the viewer's eyes preferably are oriented straight ahead at infinity (e.g, approximately centered horizontally and vertically with low vergence), the intent target behavior profile may include presenting a visual target at an apparent infinite vergence depth at least approximately centered horizontally and vertically. Similarly, when a left-to-right sweep of the eyes is desired (whether at a particular vergence depth or not) the intent target behavior profile may include moving a visual target from left to right as displayed.

Left and right stereo target fractions are established 0880. The left and right stereo target fractions are displayed 0882 per the intent target behavior profile. Certain previous examples have noted that a visual target may be presented first elsewhere, e.g., in a convenient position/configuration and then adjusted towards the intent target behavior profile. However, as also noted, it may be suitable to display 0882 the target fractions at or at least approximately at the location(s) specified by the intent target behavior profile, without first being positioned “off plan” and then being adjusted. The arrangement in FIG. 8 presents an example of such. Consequently, no step of adjusting towards the intent target behavior profile is explicitly shown in FIG. 8 (though embodiments including such adjustment also may be suitable).

The intent target behavior profile is executed 0886 with the stereo target fractions. To continue the simple example wherein the viewer is desired to look straight ahead, in such case executing 0886 the intent target behavior profile may include holding a suitable position for some predetermined length of time, until some action completes, until data is fully acquired, until the viewer or some other person gives a command, etc. However, the intent target behavior profile is not limited to such, and other (potentially much more complex) movements, positions, transformations, etc. for the stereo target fractions may be executed 0886.

An eye parameter is determined 0888. For example, a determination regarding (e.g., a measurement of) intraocular pressure may be made, an image of the eye may be taken, etc. As previously noted with regard to FIG. 7 eye movement may be tracked, and such may be considered an example of another suitable eye parameter as may be determined 0888. For example, tracking eye movement may illustrate not only whether a viewer is complying with a course of treatment (e.g., to address glaucoma), but in addition or instead may be revealing of other aspects of eye health. For example, if the user were to sweep their eyes from left to right, but the range of motion was atypically low, the speed were unexpectedly high or low, the motion exhibited jerkiness or vertical motion (when only horizontal motion is intended), such features may have diagnostic value. Regardless, whether relating to eye motion, intraocular pressure, or other eye parameters, the parameters as may be determined 0888 are not limited.

Still with reference to FIG. 8, the eye parameter as determined 0888 is registered 0892 in some fashion. As noted with regard to certain other examples herein, registration may vary considerably. Some or all of the data acquired regarding the eye parameter may be recorded in a data store, communicated to an external system, displayed, forwarded to some person, etc. The eye parameter in question may affect the form of registration 0892, e.g., a measurement of intraocular pressure may be displayed as a numerical value, while taking an eye photograph may not produce a numerical value as may be usefully displayed but may be confirmed through illuminating a telltale, etc. In addition, other information, including but not limited to the time, the patient identity, and the nature of the parameter and/or the determination thereof also may be registered 0892.

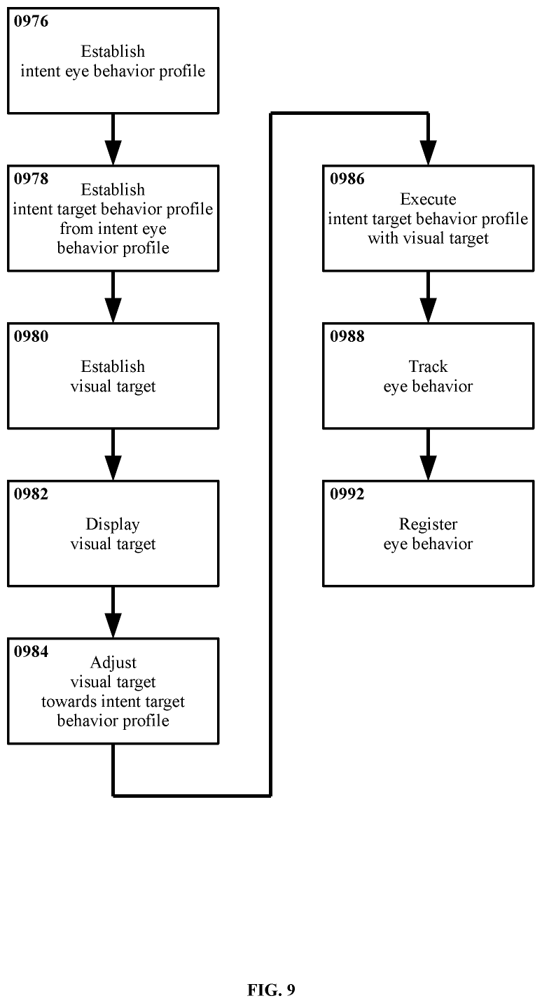

Now with reference to FIG. 9, certain previous examples herein have referred to stereo targets, e.g., with left and right stereo fractions being displayed so as to present an appearance of target depth to a viewer. However, while stereo display may be useful for certain applications, the use of non-stereo imagery, e.g., a mono target, background, etc., may be suitable in at least certain instances. (Conversely, a full-field or partial field stereo display, wherein not only the visual target but also other content may be displayed in stereo, also may be suitable.) For example, if—as referenced with regard to FIG. 8—a test were to be performed wherein the user is encouraged to sweep their eyes left to right one or more times, it may not necessarily be required that the user maintain any particular vergence, focus, etc. for such sweeps (though arrangements wherein sweeps at particular apparent depths are not prohibited). In such case a non-stereo visual target may be suitable, e.g, wherein the viewer may be presented with the same image to both eyes rather than left and right stereo fractions to the left and right eyes respectively. FIG. 9 presents an example with reference to mono content.

In FIG. 9, an intent eye behavior profile is established 0976, and an intent target behavior profile 0978 is established from the intent eye behavior profile. A visual target is established 0980. In certain previous examples herein stereo target fractions have been established, but as shown in the example of FIG. 9 the target (and/or other content to be displayed) may be mono. Mono content typically may not require a stereo display, and consequently may demand less graphical sophistication of display system, less processing power (presenting one image rather than two), etc. While stereo content is not forbidden (even in arrangements wherein the visual target itself is mono, with other stereo features also being displayed), stereo content is not necessarily required, and mono visual targets may be suitable.

Continuing in FIG. 9, the visual target is displayed 0982, and the visual target is adjusted 0984 towards the intent target behavior profile. The visual target is made to execute 0986 the intent target behavior profile, and eye behavior is tracked 0988. Eye behavior may be registered 0992, e.g., recorded in a data store, displayed, communicated, etc. The manner by which at least certain steps utilizing non-stereo content may be exhibit certain differences from the use of stereo content, e.g., executing an intent target behavior profile with stereo target fractions may involve two graphical entities following two display “flight plans” while a single mono visual target may only follow one. However, other features, and other steps altogether, may not be greatly different for mono as opposed to stereo content, for example registering eye behavior such as test results, eye motion, etc. may be similar regardless of the target displayed.

It is noted that a variety of eye related therapeutic processes, apparatuses, etc. are presented herein as examples. While certain such are presented individually for purposes of clarity, combination thereof also may be suitable. For example, an arrangement as may be similar to that in FIG. 8 may be used to facilitate eye testing of a patient; an arrangement as may be similar to that in FIG. 2 then may be used for conducting tests/observations as so facilitated, in order to obtain information about the patient's eye health; and an arrangement as may be similar to that in FIG. 6 may be used to provide intervention with the patient in the form of therapeutic treatment (presuming testing revealed a some condition for which treatment may be considered). Such an arrangement may be understood as an “end-to-end” approach, preparing the subject for eye testing, carrying out eye testing, and providing treatment for an eye condition indicated by eye testing. Other combinations and/or variations also may be suitable.

In addition, it is noted that some or all such processes and/or sub-processes may be carried out in an automated manner. For example, test preparation, testing, and/or treatment may be implemented through processor control, e.g., the activation of executable instructions instantiated on a processor. Systems likewise may be adaptive, responding to the user's performance, for example modifying, extending, reducing, adding, and/or removing functions based on test results, eye tracking, degree of adherence to a treatment regimen, etc. As a more concrete example, a system may respond to progression of glaucoma in a patient may respond by changing to a treatment regimen (e.g., eye focus, eye movement, etc.) that is more intensive, increasing treatment session length and/or frequency, or advising the patient to contact an eye care professional (or contact such a professional automatically). In addition, systems may interact more directly with patients, such as by monitoring for keywords or key phrases (“pressure in my eyes”), accepting commands through voice input or other avenues, etc. Any or all such functions may be carried out autonomously, without the need for direct routine involvement by eye care professionals. While involvement of an eye care professional or other caregiver is not excluded, neither may such involvement be required.

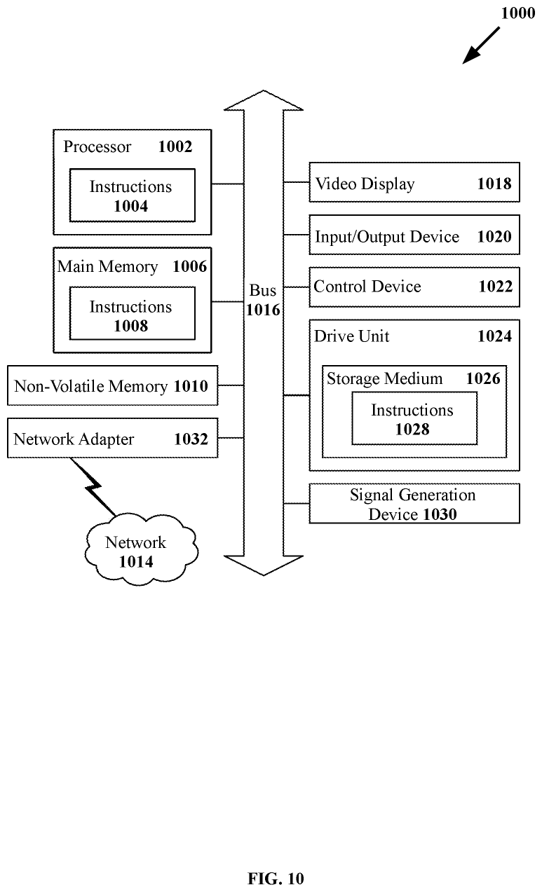

Now with reference to FIG. 10, therein is shown a block diagram illustrating an example of a processing system 1000 in which at least some operations described herein can be implemented. The processing system may include one or more central processing units (“processors”) 1002, main memory 1006, non-volatile memory 1010, network adapter 1012 (e.g., network interfaces), video display 1018, input/output devices 1020, control device 1022 (e.g., keyboard and pointing devices), drive unit 1024 including a storage medium 1026, and signal generation device 1030 that are communicatively connected to a bus 1016. The bus 1016 is illustrated as an abstraction that represents any one or more separate physical buses, point to point connections, or both connected by appropriate bridges, adapters, or controllers. The bus 1016, therefore, can include, for example, a system bus, a Peripheral Component Interconnect (PCI) bus or PCI-Express bus, a HyperTransport or industry standard architecture (ISA) bus, a small computer system interface (SCSI) bus, a universal serial bus (USB), IIC (I2C) bus, or an Institute of Electrical and Electronics Engineers (IEEE) standard 1394 bus, also called “Firewire.”

In various embodiments, the processing system 1000 operates as a standalone device, although the processing system 1000 may be connected (e.g., wired or wirelessly) to other machines. In a networked deployment, the processing system 1000 may operate in the capacity of a server or a client machine in a client-server network environment, or as a peer machine in a peer-to-peer (or distributed) network environment.

The processing system 1000 may be a server, a personal computer (PC), a tablet computer, a laptop computer, a personal digital assistant (PDA), a mobile phone, a processor, a telephone, a web appliance, a network router, switch or bridge, a console, a hand-held console, a (hand-held) gaming device, a music player, any portable, mobile, hand-held device, or any machine capable of executing a set of instructions (sequential or otherwise) that specify actions to be taken by the processing system.

While the main memory 1006, non-volatile memory 1010, and storage medium 1026 (also called a “machine-readable medium) are shown to be a single medium, the term “machine-readable medium” and “storage medium” should be taken to include a single medium or multiple media (e.g., a centralized or distributed database, and/or associated caches and servers) that store one or more sets of instructions 1028. The term “machine-readable medium” and “storage medium” shall also be taken to include any medium that is capable of storing, encoding, or carrying a set of instructions for execution by the processing system and that cause the processing system to perform any one or more of the methodologies of the presently disclosed embodiments.

Still with reference to FIG. 10, in general the routines executed to implement the embodiments of the disclosure may be implemented as part of an operating system or a specific application, component, program, object, module or sequence of instructions referred to as “computer programs.” The computer programs typically comprise one or more instructions (e.g., instructions 1004, 1008, 1028) set at various times in various memory and storage devices in a computer, and that, when read and executed by one or more processing units or processors 1002, cause the processing system 1000 to perform operations to execute elements involving the various aspects of the disclosure.

Moreover, while embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Further examples of machine-readable storage media, machine-readable media, or computer-readable (storage) media include, but are not limited to, recordable type media such as volatile and non-volatile memory devices 1010, floppy and other removable disks, hard disk drives, optical disks (e.g., Compact Disk Read-Only Memory (CD ROMS), Digital Versatile Disks, (DVDs)), and transmission type media such as digital and analog communication links.

The network adapter 1012 enables the processing system 1000 to mediate data in a network 1014 with an entity that is external to the computing device 1000, through any known and/or convenient communications protocol supported by the processing system 1000 and the external entity. The network adapter 1012 can include one or more of a network adapter card, a wireless network interface card, a router, an access point, a wireless router, a switch, a multilayer switch, a protocol converter, a gateway, a bridge, bridge router, a hub, a digital media receiver, and/or a repeater.

The network adapter 1012 can include a firewall that can, in some embodiments, govern and/or manage permission to access/proxy data in a computer network, and track varying levels of trust between different machines and/or applications. The firewall can be any number of modules having any combination of hardware and/or software components able to enforce a predetermined set of access rights between a particular set of machines and applications, machines and machines, and/or applications and applications, for example, to regulate the flow of traffic and resource sharing between these varying entities. The firewall may additionally manage and/or have access to an access control list which details permissions including for example, the access and operation rights of an object by an individual, a machine, and/or an application, and the circumstances under which the permission rights stand.

As indicated above, the computer-implemented systems introduced here can be implemented by hardware (e.g., programmable circuitry such as microprocessors), software, firmware, or a combination of such forms. For example, some computer-implemented systems may be embodied entirely in special-purpose hardwired (i.e., non-programmable) circuitry. Special-purpose circuitry can be in the form of, for example, application-specific integrated circuits (ASICs), programmable logic devices (PLDs), field-programmable gate arrays (FPGAs), etc.

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling others skilled in the relevant art to understand the claimed subject matter, the various embodiments, and the various modifications that are suited to the particular uses contemplated.

While embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Although the above Detailed Description describes certain embodiments and the best mode contemplated, no matter how detailed the above appears in text, the embodiments can be practiced in many ways. Details of the systems and methods may vary considerably in their implementation details, while still being encompassed by the specification. As noted above, particular terminology used when describing certain features or aspects of various embodiments should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless those terms are explicitly defined herein. Accordingly, the actual scope of the invention encompasses not only the disclosed embodiments, but also all equivalent ways of practicing or implementing the embodiments under the claims.

The language used in the specification has been principally selected for readability and instructional purposes, and it may not have been selected to delineate or circumscribe the inventive subject matter. It is therefore intended that the scope of the invention be limited not by this Detailed Description, but rather by any claims that issue on an application based hereon. Accordingly, the disclosure of various embodiments is intended to be illustrative, but not limiting, of the scope of the embodiments, which is set forth in the following claims.

What is claimed is:

1. A method, comprising:
determining an intent eye configuration for eyes of a viewer comprising orientations of left and right eyes, wherein said intent eye configuration corresponds to a reduction of an intraocular pressure of said eyes;
determining an intent vergence depth of a visual target corresponding with said intent eye configuration;
generating left and right stereo image fractions for said visual target;
displaying said left and right stereo image fractions so as to enable a resolution of said left and right stereo image fractions by said viewer; and
changing said vergence depth of said left and right stereo image fractions over time towards said intent vergence depth, such that responsive to maintaining said resolution of said left and right stereo image fractions by said viewer, said eyes of said viewer bias towards said intent eye configuration, so as to facilitate said reduction of said intraocular pressure of said eyes.

2. The method of claim 1, further comprising:
changing said vergence depth of said left and right stereo image fractions over time dynamically proximate said intent vergence depth, such that responsive to maintaining said resolution of said left and right stereo image fractions by said viewer, said eyes of said viewers are biased towards a dynamic eye configuration dynamically proximate said intent eye configuration, so as to facilitate said reduction of said intraocular pressure of said eyes.

3. The method of claim 1, comprising:
displaying said left and right stereo image fractions with a head mounted display; and
changing said vergence depth of said left and right stereo image fractions with a processor of said head mounted display.

4. A method, comprising:
determining an intent eye configuration for eyes of a viewer comprising an intent focus depth of lenses of said eyes, wherein said intent eye configuration corresponds to a decrease in myopia of said eyes;
determining an intent focus depth of a visual target corresponding with said intent eye configuration;
determining an intent vergence depth of said visual target corresponding with said intent focus depth;
generating left and right stereo image fractions for said visual target;
displaying said left and right stereo image fractions with a vergence depth thereto, so as to enable a resolution of said left and right stereo image fractions by said viewer; and
changing said vergence depth of said left and right stereo image fractions over time towards said intent vergence depth, such that responsive to maintaining said resolution of said left and right stereo image fractions by said viewer, said eyes of said viewers are biased towards said intent eye configuration, so as to facilitate said decrease in myopia of said eyes.

5. The method of claim 4, comprising:
determining an intent eye path of said visual target corresponding with said intent eye configuration, and determining an intent target path of said visual target corresponding with said intent eye path;
displaying said left and right stereo image fractions with a target fraction position thereto, so as to enable a resolution of said left and right stereo image fractions by said viewer; and
changing said target fraction position of said left and right stereo image fractions over time towards said intent target path, such that responsive to maintaining said resolution of said left and right stereo image fractions by said viewer, said eyes of said viewers are biased towards said intent eye configuration, so as to facilitate said decrease in myopia of said eyes.

6. The method of claim 4, comprising:
displaying said left and right stereo image fractions with a head mounted display; and
changing said vergence depth of said left and right stereo image fractions with a processor of said head mounted display.

7. A method, comprising:
determining an intent eye configuration for eyes of a viewer that corresponds to an intent test path for said eyes;
determining an intent target path of a visual target corresponding with said intent eye configuration;
determining left and right intent display positions for left and right stereo image fractions for said visual target corresponding with said intent eye configuration;
generating said left and right stereo image fractions for said visual target;
displaying said left and right stereo image fractions at display positions so as to enable a resolution of said left and right stereo image fractions by said viewer; and
changing said display positions of said left and right stereo image fractions over time towards said intent display positions, such that responsive to maintaining said resolution of said left and right stereo image fractions by said viewer, said eyes of said viewers are biased towards said intent eye configuration, so as to facilitate executing a test of said eyes;
executing said test; and
registering said test.
